(19) United States
(12) Patent Application Publication
(10) Pub. No.: US 2006/0004190 A1
(43) Pub. Date: Jan. 5, 2006

(54) Glycoside and orthoester glycoside derivatives of apomorphine, analogs, and uses thereof

(76) Inventors: Michael Holick, Sudbury, MA (US); Halasya Ramanathan, Brooklyn, NY (US)

Correspondence Address: CLARK & ELBING LLP, 101 FEDERAL STREET, BOSTON, MA, 02110, US

(21) Appl. No.: 10/508,160

(22) PCT Filed: Mar. 19, 2003
(86) PCT No.: PCT/US03/08448

§ 371 Date: Aug. 26, 2005

Related U.S. Application Data
(60) Provisional application No. 60/365,454, filed on Mar. 19, 2002.

(51) Int. Cl. A61K 31/7052; C07H 17/02

(52) U.S. Cl. 536/17.4; 514/23

(57) ABSTRACT

Disclosed are glycoside and orthoester glycoside derivatives of apomorphine and analogs thereof to treat conditions and diseasessuch as erectile dysfunction.

### BACKGROUND OF THE INVENTION

1. Field of the Invention

The present invention relates to glycosides and orthoester glycosides of apomorphine and analogs and their use in therapy.

2. Related Art

Segraves, R. T., “Dopamine agonists and their effect on the human penile erectile response, pp 225-229 in Bancroft, J., editor, The Pharmacology of Sexual Function and Sexual Dysfunction, Excerpta Medica, Amsterdam (1995), discloses that dopamine agonists, including apomorphine, induce erectile responses in the human male.

U.S. Pat. No. 5,744,476 discloses the use of dopamine D₁ agonists for the treatment of senile dementia and dementia associated with neurodegenerative diseases such as Parkinson's disease and Alzheimer's disease.

U.S. Pat. No. 5,756,483 discloses pharmaceutical compositions for intranasal administration of apomorphine, a very powerful dopamine agonist useful for the treatment of Parkinson's disease, complicated by motor fluctuations. According to the '483 patent, the compositions comprising cyclodextran and/or other saccharides and/or sugar alcohols exhibit high bioavailability and stability of apomorphine.

U.S. Pat. Nos. 5,770,606, 5,985,889, 6,121,276, 6,200,983 and 6,306,437 disclose the sublingual administration of apomorphine to ameliorate, without substantial undesirable side effects, pyschogenic impotence or erectile dysfunction. Such side effects include nausea, hypertension, flushing and diaphoresis. The patents also teach that apomorphine has poor oral bioavailability. Also disclosed is the oral administration of apomorphine and an antiemetic agent to substantially reduce nausea.

U.S. Pat. No. 5,888,534 discloses the controlled release of apomorphine by sublingual or buccal administration for the treatment of pyschogenic impotence and Parkinson's disease.

U.S. Pat. No. 5,939,094 discloses dosage forms for the transdermal administration of apomorphine for the treatment of Parkinson's disease.

U.S. Pat. No. 5,994,363 discloses the treatment of Parlcinson's disease and psychogenic erectile dysfunction and the amelioration of apomorphine adverse effects such as nausea, vomiting, yawning, and cardiovascular effects, by a dose escalating method of acclimatization.

U.S. Pat. No. 5,945,117 discloses the treatment of female sexual dysfunction, without substantial undesirable side effects, by sublingual administration of apomorphine dosage forms. Administration of apomorphine increases nerve stimulated clitoral intracavemosal blood flow and vaginal wall blood flow for enhanced clitoral erection and vaginal engorgement in a female. A plasma concentration of apomorphine of no more than about 5.5 nanograms per milliliter is preferably maintained.

U.S. Pat. No. 6,001,845 discloses a method of treating sexual dysfunction comprising administering a therapeutically effective amount of a combination of phentolamine or a salt, solvate, hydrate, or crystalline polymorph thereof and apomorphine or a salt, solvate or hydrate thereof. The two drugs may be administered either substantially concurrently in separate dosage forms or combined in a single unit dosage form. See also U.S. Pat. No. 6,011,043.

U.S. Pat. No. 6,087,362 discloses a method for the treatment of sexual dysfunction in human patients by administering orally apomorphine and sidenafil. According to the '362 patent, the combination optimizes the efficacy of each drug and minimizes the undesirable side effects associated with the individual drugs. The drugs may be coadministered in a combination dosage form or administered sequentially in separate dosage forms prior to sexual activity. Also according to the '362 patent, antiemetic agents such as antidopaminergic agents (e.g. benzamides such as metaclopramide, trimethobenzamide, benzquinamid), phenothiazines (e.g. chlorpromazine, prochlorperazine, pipamazine, thienylperazine, oxypendyl hydrochloride, promazine, triflupromazine, propiomazine, acepromazine, acetophenazine, butaperazine, carpherazine, fluphenazine, perphenazine, thiopropazate, trifluoperazine, mesoridazine, peperacetazine, thioridazine, pepotiazine, pepotiazine palmitate, chlorprothixine, doxepin, loxapin, triflupromazine, methdilazine, trimeprazine, and methotrimeprazine), serotonin (5-hydroxytriptamine or 5-HT) antagonists (e.g. domperidone and odansetron), histamine antagonists (e.g. buclizine hydrochloride, cyclizine hydrochloride and dimenhydrinate), parasympathetic depressants (e.g. scopolamine), other antiemetics (e.g. metopimazine, trimethobenzamide, benzoquinamine hydrochloride and diphenidol hydrochloride), and piperazines (e.g. meclizine and chlorcyclizine) may be coadministered.

U.S. Pat. No. 6,136,818 discloses administering a combination of phentolamine and apomorphine for the treatment of human sexual function and dysfunction.

U.S. Pat. No. 6,266,560 discloses a method for enhancing erectile function by applying an electric pulse to the penis and substantially contemporaneously applying a vasoactive or androgenic composition thereto. Examples of vasoactive compounds include apomorphine.

U.S. Pat. No. 6,291,471 discloses a method of treating male organic erectile dysfunction having a vasculogenic origin by orally administering an effective amount of apomorphine or a salt thereof.

U.S. Pat. No. 6,316,027 discloses fast dissolving dosage forms comprising dopamine agonists such as apomorphine, water, gelatin and other ingredients for the treatment of Parkinson's disease.

### SUMMARY OF THE INVETION

The present invention relates to a pro-drug approach to apomorphine therapy that provides better bioavailability, less emetic action and allows oral administration. The pro-drug is in the form of glycosides and orthoester glycosides of apomorphine and analogs thereof. The catechol moiety in apomorphine may be glycosylated cleanly to give one major isomer. When administered, glycosidase enzymes in the biological medium of human body cleave the glycoside/orthoester glycoside, liberating the free drug. Thus the free drug is bioavailable in a controlled fashion as determined by the rate of deglycosylation.

Apomorphine is a dopamine receptor agonist that acts on the central nervous system. Once absorbed and transported into the brain, apomorphine initiates a chain of reactions that result in increased blood flow to the male genital organs and an erection. Thus, apomorphine and its glycosides/ortho ester glycosides derivatives can be used to treat sexual dysfunctions, Parkinson's disease and other conditions treatable with apomorphine.

In a first aspect, the present invention provides a composition for the treatment of a condition treatable by the administration of apomorphine or an analog thereof, characterized in that the apomorphine or analog thereof is a derivative in the form of a glycoside or orthoester glycoside, derivative or salt or ester of the derivative.

The present invention also relates to compounds of the Formula (I):
Apo-(OR₁)ₙ (I)

or a salt or ester thereof;

wherein Apo is an apomorphine residue or analog thereof, n is 1 or 2, and R₁ is

a straight or branched chain glycosidic moiety containing 1-20 glycosidic units,

or R₁ is an orthoester glycoside moiety of the Formula (II):

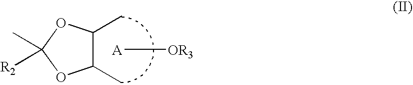

wherein A represents a glycofuranosyl or glycopyranosyl ring;

R₂ is hydrogen or alkyl;

R₃ is hydrogen or a straight or branched chain glycosidic moiety containing 1-20 glycosidic units; or

when n is 2, both R₁ groups form a ketal or acetal having the Fomula (III):

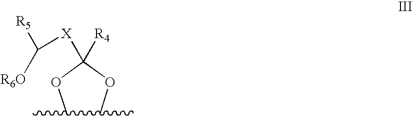

wherein X is a straight or branched chain alkylene group;

R₄ and R₅ are independently hydrogen or an alkyl group; and

R₆ is a straight or branched chain glycosidic moiety containing 1-20 glycosidic units or an orthoester glycoside as defined above.

In a preferred embodiment, the compound has one of Formulae (IV), (V), or (VI) or (VII): 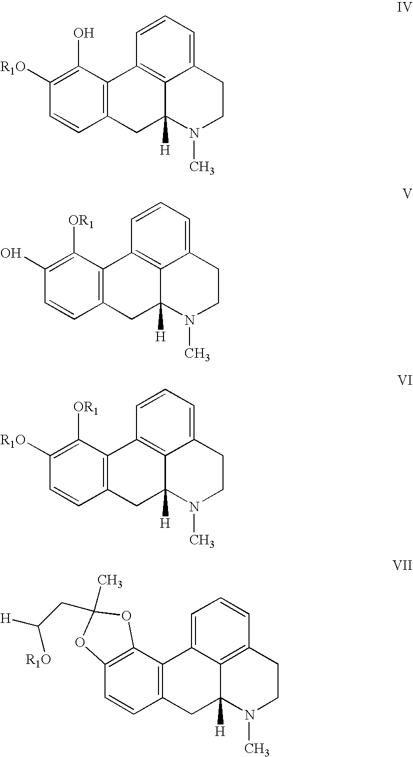
or a salt, or ester thereof, wherein R₁ is defined above.

The invention also relates to a method for the treatment or amelioration of senile dementia and dementia associated with neurodegenerative diseases such as Alzheimer's disease and other disorders involving memory loss and/or dementia (including AIDS dementia); disorders of attention and focus (such as attention deficit disorder); disorders of extrapyramidal motor function such as Parkinson's disease, Huntington's disease, Gilles de la Tourette syndrome and tardive dyskinesia; mood and emotional disorders such as depression, panic, anxiety and psychosis treatment or amelioration of erectile dysfunction or female sexual dysfunction, comprising administering to an animal in need thereof, an effective amount of a compound having the Formulae (I), (IV), (V), (VI) or (VI), or a pharmaceutically acceptable salt or ester thereof.

The invention also relates to a method of preparing a compound of Formulae (I), (IV), (V) and (VI) which comprises reacting a protected α-bromoglycoside or orthoester glycoside with apomorphine or analog thereof in the presence of a base and cleaving the protecting groups. The desired compound may be isolated from a mixture of products either before or after the protecting groups are cleaved.

In a further embodiment, apomorphine or analog thereof may be glycosylated with the glycosylation donor 1-trichloroacetarnidoyl-2,3,4,6-tetra-O-acyl-glycopyranoside in an aprotic solvent under an inert atmosphere. A Lewis acid such as boron trifluoride etherate is added, the reaction stirred and the product isolated. Cleavage of the acyl protecting groups gives the apomorphine glycoside.

In another embodiment, the catechol moiety of apomorphine is lcetalyzed with an aldehyde or ketone having the Formula (VIII): 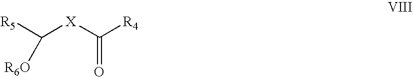
wherein X, R₄ and R₅ are defined above and the hydroxy groups on R₆ are protected. Removal of the protecting groups gives a compound of Formula I.

By “apomorphine analog” is meant a dopamine receptor agonist of formulas IX or X: 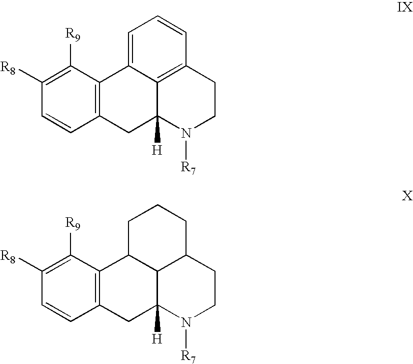
wherein R₇ is hydrogen, alkyl, halogen substituted alkyl, hydroxyl substituted alky, aryl substituted alkyl, acyl substituted alkyl, acyl, or aryl; R₈ and R₉ are each independently selected from hydrogen, hydroxyl, alkyl, sulfhydryl, halogen, —O-alkyl, and —O-acyl, provided that at least one of R₈ and R₉ is a hydroxy group; and each methane and methylene proton of formulas IX and X are optionally substituted by halogen, nitro, —NH₂, secondary amino, tertiary amino, quaternary amino, —S-alkyl, —S-acyl, sulfhydryl, hydroxyl, alkyl, —O-alkyl, —O-acyl, halogen substituted alkyl, hydroxyl substituted alkyl, aryl substituted alkyl, and acyl substituted alkyl. Furthermore, apomorphine analogs include those compounds disclosed in U.S. Pat. Nos. 4,120,964; 4,353,912; 4,543,256; and 6,313,134, hereby incorporated by reference. Exemplary apomorphine analogs include (R)—N-n-propylnorapomorphine, (R)—N-methyl-10-hydroxyhexahydroaporphine, (R)-11-hydroxy-10-methyl aporphine, and (R)-11-hydroxy-N-n-propylnoraporphine.

### DETAILED DESCRIPTION OF THE INVENTION

Where the derivative is a glycoside, then it is preferred that it contain 1-20 glycosidic units.

It is preferred that compounds of the present invention have less than 10 and, more preferably, 3 or less glycosidic units. Specific examples are those containing 1 or 2 glycosidic units in the glycoside moiety, such as glucose and sucrose, with one being most preferred.

By glycosidic units are meant glycopyranosyl or glycofuranosyl, as well as their sulfates, amino sugar and/or deoxy derivatives. The configuration of each unit may be D or L, although D is generally preferred. The moieties may be homopolymers, random or alternating polymers, or block copolymers of these monomers.

The glycosidic units have free hydroxy groups, or the hydroxy groups may be acylated, e.g. with a group R₄—(C═O)—, wherein R₄ is hydrogen, C₁₋₆ alkyl, C₆₋₁₀ substituted or unsubstituted aryl or C₇₋₁₆ arakyl. Preferably, the acyl groups are acetyl or propionyl. Other preferred R₄ groups are phenyl, nitrophenyl, halophenyl, lower alkyl substituted phenyl, lower alkoxy substituted phenyl and the like or benzyl, lower alkoxy substituted benzyl and the like.

The glycopyranose or glycofuranose ring or amino derivative thereof may be fully or partially acylated or completely deacylated. The completely or partially acylated glycoside is useful as a defmed intermediate for the synthesis of the deacylated material. Useful protecting groups include, but are not limited to, acetyl, benzoyl, nicotinoyl, benzyl, methyl and phenyl.

Among the possible glycopyranosyl structures are glucose, mannose, galactose, gulose, allose, altrose, idose, or talose. Among the furanosyl structures, the preferred ones are derived from fructose, ribose, arabinose or xylose. Among preferred diglycosides are sucrose, cellobiose, maltose, lactose, trehalose, gentiobiose, and melibiose. Among the triglycosides, the preferred ones may be raffmose or gentianose.

Preferred aminosugar derivatives are N-acetyl-D-galactosamine, N-acetyl-D-glucosamine, N-acetyl-D-mannosamine, N-acetylneuraminic acid, D-glucosamine, D-lyxosylamine, D-galactosamine, chondroitin, and the like. In addition, such active units as chondroitin sulfate and D-glucosamine sulfate may also be employed, as such sub-units independently have advantageous therapeutic osteopathic properties.

Where there are linked glycosidic units, i.e., there is a di or polyglycosidic moiety, the individual glycosidic rings may be bonded by 1-1, 1-2, 1-3, 1-4, 1-5 or 1-6 bonds, most preferably 1-2, 1-4 and 1-6. The linkages between individual glycosidic rings may be α or β.

Alkyl groups may be straight, branched or cyclic and may conveniently be a C₁₋₁₀ alkyl, including octyl, nonyl, decyl, diethylhexyl, and, more preferably, C₁₋₆, such as methyl, ethyl, propyl, butyl, methylpropyl, t-butyl, pentyl, dimethylpropyl, hexyl, dimethylbutyl or ethylbutyl. Preferred alkyl groups contain 1 or 2 carbon atoms. Methyl and ethyl groups are particularly preferred, especially methyl.

Straight and branch chain alkylene groups include C₁₋₆ alkylene groups optionally substituted with one or more alkyl groups.

Aryl groups generally have 6 to 14 carbon atoms having a single ring (e.g., phenyl) or multiple condensed rings (e.g., naphthyl or anthryl). Preferred aryl groups are phenyl and naphthyl, preferably phenyl.

Especially preferred apomorphine derivatives include, without limitation, those with Formulae (IV)-(VII), wherein R₁ is a glucosyl moiety.

The compounds useful in the practice of the invention contain at least one glycoside or orthoester glycoside moiety connected to the 10- and/or 11-hydroxyl group of apomorphine or analog thereof.

The water soluble glycosidic derivatives of the aforementioned apomorphine and analogs thereof may be obtained according to the general methods disclosed U.S. Pat. No. 4,410,515, the contents of which are fully incorporated by reference herein.

Salts of the compounds of the invention include any pharmaceutically acceptable salts include the acid addition salts with e.g. hydrogen chloride, sulfuric acid, phosphoric acid, acetic acid, malic acid, carbonic acid and the like.

Esters of the compounds of the invention include esters of any free hydroxy groups on apomorhine and analogs thereof. Such esters include the group R₄—(C═O)—, wherein R₄ is as defined above.

The invention is related in particular to the synthesis of compounds of Formula (I). In one embodiment, apomorphine or analog thereof may be glycosylated with the glycosylation donor 1-trichloroacetamidoyl-2,3,4,6-tetra-O-acyl-glycopyranoside in an aportic solvent under an inert atmosphere. Examples of such acyl groups include R₄—(C═O)— defined herein above. Especially preferred acyl groups are acetyl groups. Examples of aprotic solvents include dichlormethane, chloroform and the like. The reaction is stirred at ambient temperature and a Lewis acid such as boron trifluoride etherate is added, the reaction stirred and the product isolated. Isolation may be accomplished with any conventional method such as column chromatography on silica gel. Cleavage of the acyl protecting groups gives the apomorphine glycoside. When the protecting groups are C₂₋₆ alkanoyl, they may be removed by any known methods including treatment with alkali allcoxide in alcohol (e.g., sodium methoxide in methanol) or by treatment with a basic resin in alcohol (e.g., DOWEX 110-OH in methanol). In the case of trialkylsilyl and aryldialkylsilyl protecting groups, they may be removed in the presence of fluoride (e.g., tetrabutylammonium fluoride). In the case of benzyl groups, they may be removed by hydrogenation.

In a second embodiment, a protected α-bromoglycoside or orthoester glycoside is reacted with apomorphine or an analog thereof in the presence of a base and the protecting groups are cleaved. The desired compound may be isolated from a mixture of products either before or after the protecting groups are cleaved.

Examples of bases that can be used to form the desired linkage between apomorphine or an analog thereof and a bromoglycoside or orthoester glycoside include cadmium carbonate, silver carbonate, silver silicate, barium carbonate, lanthanum carbonate or oxalate, ytterbium carbonate or oxalate, and uranium carbonate or oxalate. In some cases, the glycoside or orthoester glycoside incorporates protecting groups. Examples of protecting groups include C₂₋₆ alkanoyl groups (e.g., the peracetate) and trialkylsilyl groups (e.g., t-butyldimethylsilyl and triisopropylsilyl). The reaction is carried out in an aprotic solvent, such as benzene, toluene, tetrahydrofuran, xylenes, chlorobenzene, dichlorobenzenes and the like.

The reaction temperature is from about 80 to 120 C. Preferably, the reaction temperature is about 110 C.

The reaction may be carried out for about 1 to 18 hours, preferably, about 4 hours or until TLC shows that the reaction is complete.

The thus formed apomorphine or analog thereof-glycoside or orthoester glycoside is then isolated and may be purified on a silica gel column. The protecting groups can then be removed and the glycoside/orthoester glycoside can be isolated and purified.

Representative examples of diseases and conditions treatable by compounds of the present invention are as listed hereinabove, and include, but are not limited to, erectile dysfunction, female sexual dysfunction, senile dementia and dementia associated with neurodegenerative diseases such as Alzheimer's disease and other disorders involving memory loss and/or dementia (including AIDS dementia); disorders of attention and focus (such as attention deficit disorder); disorders of extrapyramidal motor function such as Parkinson's disease, Huntington's disease, Gilles de la Tourette syndrome and tardive dyskinesia; mood and emotional disorders such as depression, panic, anxiety and psychosis.

Benign Prostatic Hyperplasia (BPH) has been associated with erectile dysfunction (ED). Apomorphine is known to be useful for treating for ED in patients with BPH. Thus, the compounds of the invention are useful for treating and ameliorating ED associated with BPH.

Coronary artery disease (CAD) is also associated with ED. Thus, the compounds of the invention are also useful for treating and ameliorating ED associated with CAD.

Particularly preferred routes of administration of the compounds of the present invention are per os, such as elixirs, tablets and capsules, as exemplified below.

More generally, the compounds of the present invention can be administered in any appropriate pharmaceutically acceptable carrier for oral administration, since the apomorphine and apomorphine analog glycoside/orthoester glycoside derivatives are biologically active upon oral administration. The compounds of the invention may also be administered in any appropriate pharmaceutical carrier for parenteral, intramuscular, transdermal, intranasal, buccal or inhalation administration. They can be administered by any means that treat or ameliorate erectile dysfunction, female sexual dysfunction, senile dementia and dementia associated with neurodegenerative diseases such as Alzheimer's disease and other disorders involving memory loss and/or dementia (including AIDS dementia); disorders of attention and focus (such as attention deficit disorder); disorders of extrapyramidal motor function such as Parkinson's disease, Huntington's disease, Gilles de la Tourette syndrome and tardive dyslidnesia; mood and emotional disorders such as depression, panic, anxiety and psychosis, ED associated with BPH and ED associated with CAD.

The compounds of the invention may also be administered by a dose escalating method of acclimatization as described in U.S. Pat. No. 5,994,363 thereby ameliorating any apomorphine adverse effects.

The compounds of the invention may also be administered by applying an electric pulse to the penis as described in U.S. Pat. No. 6,266,560 and substantially contemporaneously applying a composition of the invention thereto, thereby inducing an erection.

The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 1.0 mg to 100 mg daily of the glycoside/orthoester glycoside, in one or more dosages per day, is effective to obtain the desired results. One of ordinary skill in the art can determine the optimal dosages and concentrations of active compounds with only routine experimentation.

The compounds can be employed in dosage forms such as tablets and capsules for oral administration. Such dosage forms may comprise well know pharmaceutically acceptable carriers and excipients. In a preferred embodiment, the dosage forms comprise cyclodextran and/or other saccharides and/or sugar alcohols. The compounds may also be formulated in a sterile liquid for formulations such as solutions or suspensions for parenteral use. A lipid vehicle can be used in parenteral administration. The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Methods well known in the art for making formulations are found, for example, in “Remington: The Science and Practice of Pharmacy” (20th ed., ed. A. R. Gennaro A R., 2000, Lippincott Williams & Wilkins).

The compounds may also be employed in fast dissolving dosage forms, as described in U.S. Pat. No. 6,316,027, hereby incorporated by reference, comprising the compounds of the invention, water, gelatin and other ingredients.

Topical formulations for transdernal, intranasal or inhalation administration may be prepared according to methods well known in the art. For topical administration, the compounds may be applied in any of the conventional pharmaceutical forms. For example, the compounds may be administered as part of a cream, lotion, aerosol, ointrnent, powder, drops or transdermal patch. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight of the active compounds.

The compounds may be administered together with therapeutically effective amounts of other compounds such as yohimbine, antiemetic agents such as antidopaminergic agents (e.g. benzamides such as metaclopramide, trimethobenzamide, benzquinamid), phenothiazines (e.g. chlorpromazine, prochlorperazine, pipamazine, thienylperazine, oxypendyl hydrochloride, promazine, triflupromazine, propiomazine, acepromazine, acetophenazine, butaperazine, carpherazine, fluphenazine, perphenazine, thiopropazate, trifluoperazine, mesoridazine, peperacetazine, thioridazine, pepotiazine, pepotiazine palmitate, chlorprothixine, doxepin, loxapin, triflupromazine, methdilazine, trimeprazine, and methotrimeprazine), serotonin (5-hydroxytriptamine or 5-HT) antagonists (e.g. domperidone and odansetron), histamine antagonists (e.g. buclizine hydrochloride, cyclizine hydrochloride and dimenhydrinate), parasympathetic depressants (e.g. scopolamine), other antiemetics (e.g. metopimazine, trimethobenzamide, benzoquinamine hydrochloride and diphenidol hydrochloride), and piperazines (e.g. meclizine and chlorcyclizine). Such combinations optimize the efficacy of each drug and minimize the undesirable side effects associated with the individual drugs. The drugs may be coadministered in a combination dosage form or administered sequentially in separate dosage forms, e.g. prior to sexual activity.

In a preferred embodiment, the compounds of the invention are administered together with sildenafil, or a pharmaceutically acceptable salt or glycoronide (e.g. glucoronidea and galactoronides) thereof. Such glycoronides may be prepared by acylation of the sildenafil amide with, e.g. a hydroxy group protected active ester form of the glycoronide or with a protected glycuronolactone (e.g. a protected glucuronolactone) as described in U.S. Pat. Nos. 5,977,326 and 4,774,230, each of which is hereby incorporated by reference. Cleavage of the protecting groups gives sildenafil glycoronide. The glucose transporters within the neuro cells may facilitate the uptalce of the glycoronide conjugate before sildenafil is metabolized to inactive substances. The glycoronide conjugate releases the sildenafil by the action of amnidases or glucosidases in a controlled fashion and also increases the biological stability of the sildenafil by increasing its half life.

The compounds of the invention may also be administered together with phentolamine and salts thereof (e.g. phentolamine mesylate), as well as glycosides and orthoester glycosides of phentolamine. Such glycosides and orthoester glycosides are prepared by derivatizing the hydroxy group of phentolamine with a glycoside or orthoester glycoside as described herein. In an alternative embodiment, a glycuronic ester, e.g., glucuronic acid ester of phentolamine may be prepared and administered.

The compounds of the invention may also be administered together with alprostadil, as well as glycosides, orthoester glycosides, glycuronides and amino sugar conjugates thereof. Alprostadil has the formula:

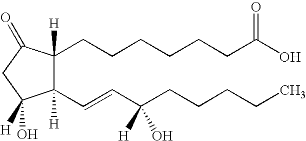

The glycosides and orthoester glycosides may be prepared by conjugation of protected a-bromoglycosides and orthoester glycosides with lower alkyl esters of alprostadil. Alternatively, alprostadil may be conjugated with 1-trichloroacetamidoyl-2,3,4,6-tetra-O-acyl-glycopyranoside. Soponification of the ester group and removal of the protecting groups on the sugar residue(s) gives the glycosides and orthoester glycosides of alprostadil. The amino sugar conjugates may be prepared by conjugation of the carboxylic acid of alprostadil with a protected amino sugar such as glucosamine peracetate. In addition, one or both of the free hydroxyl groups may be derivatized with glycosides and orthoester glycosides as described herein. Glycoronides may be prepared by condensing the alkyl ester of alprostadil with a protected glycuronolactone. See U.S. Pat. Nos. 5,977,326 and 4,908,927, or by an acid catalyzed conjugation reaction with a glucuronate ester according to U.S. Pat. No. 5,621,087, each of which is hereby incorporated by reference.

Alprostadil may be applied topically and, in females, it may be administered urethrally. Such glycosides, orthoester glycosides, and amino sugar conjugates of alprostadil provide better permeation through the skin and better pharmocokinetic profiles compared to alprostadil.

The compounds of the invention are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, although the invention is not intended to be so limited.

Having now generally described this invention, the same will be understood by reference to the following examples which are provided herein for purposes of illustration only and are not intended to be limiting unless otherwise specified.

### EXAMPLE 1

### Synthesis of R(−)-10-β-D-glucopyranosyl-, 11-hydroxyaporphine(apomorphine glucoside)

Preparation of Apomorphine:

Apomorphine hydrochloride was purchased from Sigma/Aldrich and was used as such. 1-Trichloro acetamido glucose tetra acetate was made from glucose pentaacetate. Glucose pentaacetate and Boron trifluoride etherate was bought from Aldrich.

Conversion of 10,11-Dihydroxyaporphine hydrochloride to 10,11-dihydroxyaporphine (free base):

Apomorphine as free base is prone to oxidation rapidly and also it is light sensitive. By following the following method, apomorphine can be obtained as a pure white solid.

5 grams of apomorphine hydrochloride was suspended in sonicated argon purged water (500 mL) and a saturated sodium bicarbonate solution (100 mL) was added in one lot under argon. The sodium bicarbonate solution was prepared freshly, filtered and argon purged to avoid discoloring the product. The neutralized solution was stirred for 30 minutes and ether extracted (3×100 mL). The combined ether layer was washed with water (100 mL) once and dried with magnesium sulfate. Upon evaporation of ether under reduced pressure, apomorphine free base was obtained as colorless crystals quantitatively.

Proton NMR spectrum of apomorphine free base in CDCl₃ δ 8.2-7 (multiplets; Ar—H; 5 H), δ 3.2-2.4 (multiplets, 7-aliphatic-H) and δ 2.5 (singlet; N—CH₃; 3-H)

### Preparation of 1-Hydroxy-2,3,4,6-tetra-O-acetyl-D-glucopyranoside

Glucose penta-acetate (78 g, 0.2 mole) was dissolved in tetrahydrofuran (250 ml, HPLC grade) and purged with argon. Benzyl amine (25.7 g, 0.22 mole) was added at room temperature. The mixture was stirred at room temperature for 12 hours. Tetrahydrofuran was removed by rotary evaporator below 40 C. Dichloromethane (400 mL) and ice cold dilute hydrochloric acid (1% solution in water, 500 mL) were added to the above mixture and the aqueous layer was washed once with dichloromethane (100 mL). Combined organic extracts were washed once with water and saturated sodium bicarbonate solution (100 mL each). The Organic layer was dried and evaporated. Connected to the pump to remove traces of dichloromethane. The syrup was used as such in the next step. TLC examination showed that the pentaacetate has been hydrolyzed to a polar anomeric hydroxy group (using 40% ethyl acetate and hexane mixture; staining was done with 10% sulfuric acid).

### Preparation of 1-Trichloro acetamidoyl-2,3,4,6-tetra-O-acetyl-glucopyranoside

The syrup obtained above was dissolved in dichloromethane (350 mL) and potassium carbonate (20 g, powdered) was added. The mixture was stirred magnetically and molecular sieves (50 g) were added. The mixture was stirred for 10 minutes and trichloro acetonitrile (50 g) was added. The mixture was stirred at room temperature for 14 hours. The mixture was filtered. Upon evaporation of solvents and excess trichloro acetonitrile, the desired chloroimidate was crystallized from by adding ether (150 mL). The product weighed 47 g as a colorless white powder.

Proton NMR spectrum in CDCl₃ showed δ 8.7 (singlet, NH, 1-H); δ 5.95 (singlet, anomeric β-H; 1-H); δ 5.3-3.9 (multiplets, remaining 6-H glucosyl-H) 2.1-2.0 (overlapping singlets, 4×3-acetate-H)

### Synthesis of R(−)-10 -(β-D-2′,3′,4′,6′-tetra-0-acetyl glucopyranosyl-}, 11-hydroxyaporphine(apomorphine glucoside tetraacetate)

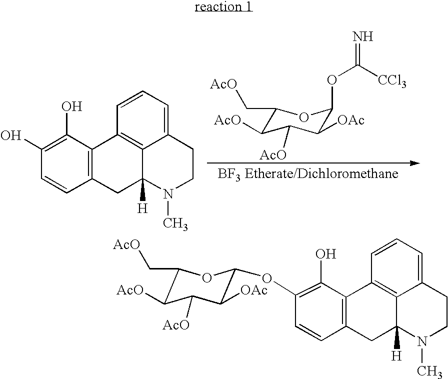

As shown in reaction 1 above, apomorphine (1.069 g; 4 mMol) in dry dichloromethane (5 mL) was stirred under argon and molecular sieves (15 g) followed by glucosylating donor 1-trichloroacetamidoyl-2,3,4,6-tetra-O-acetyl-glucopyranoside (2.452 g; 5 mMol) were added. The mixture was stirred under argon atmosphere at 20 to 25° C. for 20 minutes. Boron trifluoride etherate (0.560 mL; 4.1 mMol) was added. The progress of the reaction was monitored by TLC using ethylacetate-hexane mixtures. The reaction was essentially complete within 45 minutes. The reaction was worked up by filtering off the molecular sieves and adding the organic portion to chilled saturated sodium bicarbonate solution (100 mL) and extracting the aqueous layer once with dichloromethane (100 mL). The combined organic portion was dried over magnesium sulfate and evaporated. The product weighed 2.8 g.

Examination of the crude glucosylated product revealed that it is essentially a single product characterized by 4 doublets centered at δ 8.2, 7.1, 6.8 and 6.7 respectively (due to ortho coupling) and a multiplet at δ 7.2 due to the double ortho coupling for the five unquivalent aromatic protons. There were satellite peaks, which correspond to less than 10% of the major isomer. The above NMR pattern was identical to the starting apomorphine. The major 10-O-glucosylated-aporphine product was purified by SiO₂ column for obtaining authentic titled material. The flash SiO₂ column was eluted with ether, chloroform and methanol mixtures. The tetra-O-acetyl glucopyronosylated apomorphine eluted first. The product was isolated as a white powder soluble in most of the organic solvents.

The proton NMR spectrum was recorded in CDCl₃. δ 7.9 (doublet, Ar—H, 1-H); δ 7.3-6.7 (multiplets, Ar—H, 4-H); δ 5-3.6 (multiplets, glucosyl-H, 7 H); δ 2.2-1.9 (overlapping singlets, acetyl-H and aliphatic 1-H, 13 H), δ 3.3-2.5 (multiplets, aliphatic-H and N—CH3 singlet, 9-H).

### Synthesis of R(−)-10-{β-D-glucopyranosyl-}, 11-hydroxyaporphine(apomorphine glucoside)

Apomorphine (1.069 g, 4 mMol) and trichloro acetimidoyl 2,3,4,6-tetra-O-acetyl glucopyranoside (2.452 g, 5 mMol) in dry dichloromethane (50 mL) under argon atmosphere was stirred at 20° C. Boron trifluoride etherate (0.56 mL) was added in small portions over 10 minutes and the mixture was allowed to stir for 45 minutes. The product mixture was poured into saturated sodium bicarbonate solution (100 mL) and extracted with 2×100 mL of dichloromethane. The combined organic layers were washed with water (50 Ml) and evaporated. The crude residue was dissolved in methanol (75 mL) and Dowex-550-OH resin (10 g, without pretreatment) was added and the mixture refluxed for 2 hours. The resin was filtered off and washed once with methanol (20 mL) and evaporated. The crude gum crystallized from ethyl acetate/methanol mixtures as pale pinkish- white crystals (1.3 g)

The proton NMR spectrum was recorded in CD₃OD. δ 8.45 (doublet, 1-H, Ar—H); δ 7.25-7 (multiplet, Ar—H, 4-H); δ 5.3 (doublet, 7.2 Hz anomeric coupling, beta glucosidic linkage, 1-H); δ 3.9 to 3.0 (complex, aliphatic-H and sugar-H, 11 H); δ 2.75 (doublet, benzylic-H, 1 H); δ 2.6 (singlet, N—CH₃, 3-H) and δ 2.4 (triplet, benzylic-H, 1-H)

Mass spectrum: The molecular ion was obtained at 430.1 amu (theoretical value is 429.46 amu) in correspondence to the assigned structure.

To assign the mono glucosylation of apomorphine to either 10 or 11 position of the apomorphine nucleus, apocodeine was bought from Sigma and glucosylated. Apocodeine has methyl in 10 positions and the glucosylation under the above conditions rendered only the starting material signifying that the 11 position is more hindered for glucosylation. Thus the assigned structure for apomorphine glucoside (viz the glucosylation at the 10 position) is satisfactory.

The reaction described above yields one major product and two minor products. Structures of the reaction products are provided below.

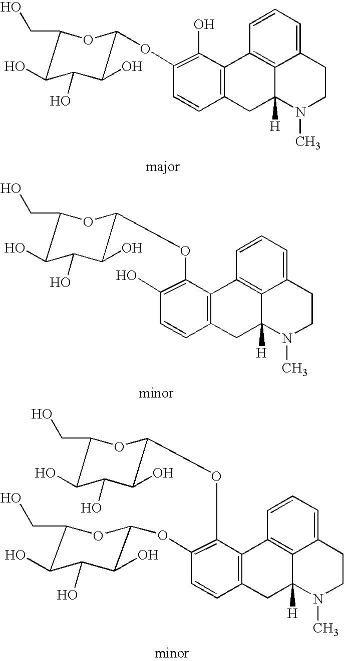

### Other Embodiments

All publications and patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. A glycoside or orthoester glycoside derivative of apomorphine or analog thereof, or salt or ester thereof.

2. The derivative of claim 1, having the Formula (I):
Apo-(OR₁)ₙ (I)
or a salt or ester thereof;
wherein Apo is an apomorphine residue or analog thereof,
n is 1 or 2, and
each R₁ is independently a straight or branched chain glycosidic moiety containing 1-20 glycosidic units, or R₁ is an orthoester glycoside moiety of the formula (II): 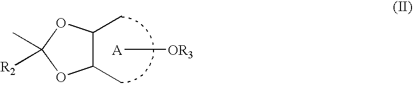
wherein A represents a glycofuranosyl or glycopyranosyl ring;
R₂ is hydrogen or alkyl;
R₃ is hydrogen or a straight or branched chain glycosidic moiety containing 1-20 glycosidic units; or
when n is 2, both R₁ groups form a ketal or acetal having the Fomula (III): 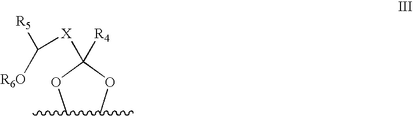
wherein X is a straight or branched chain alkylene group;
R₄ and R₅ are independently hydrogen or an alkyl group; and
R₆ is a straight or branched chain glycosidic moiety containing 1-20 glycosidic units or an orthoester glycoside as defined above.

3. The derivative of claim 1, having any one of formulas (I), (V), (VI) or (VII): 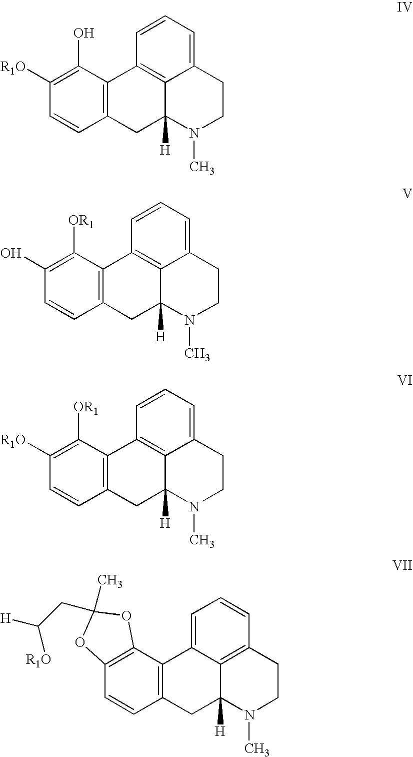
or a salt, or ester thereof, wherein R₁ is defined above.

4. The derivative of claim 1, wherein said glycoside is a moiety containing 1-20 glycosidic units.

5. The derivative of claim 1, wherein said glycoside is a glycosidic orthoester having the Formula (II): 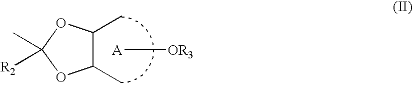
wherein A represents a glycofuranosyl or glycopyranosyl ring;
R₂ is hydrogen or alkyl;
R₃ is hydrogen or a straight or branched chain glycosidic moiety containing 1-20 glycosidic units.

6. The derivative of claim 1, wherein said glycoside is a monoglycoside.

7. The derivative of claim 6, wherein said monoglycoside is a glucoside.

8. The derivative of claim 1, wherein said glycoside derivative of apomorphine is (−)-10-{β-D-glucopyranosyl-}, 11-hydroxy aporphine.

9. A pharmaceutical composition comprising the derivative of claim 1 and a pharmaceutically acceptable carrier.

10. A method for the treatment or amelioration of sexual dysfunction in a human in need thereof, comprising administering to said human an effective amount of the derivative of claim 1.

11. The method of claim 10, wherein said dysfunction is erectile dysfimction.

12. The method of claim 10, wherein said dysfunction is female sexual dysfunction.

13. A method for the treatment or amelioration of Alzheimer's disease and other disorders involving memory loss and/or dementia; disorders of attention and focus; disorders of extrapyramidal motor function; mood and emotional disorders; comprising administering to an animal in need thereof, an effective amount of the compound of claim 1.

14. The method of any one of claims 10-13, wherein said derivative is administered as part of a pharmaceutical composition comprising a pharmaceutically acceptable carrier therefor.

15. The method of any one of claims 10-13, wherein said derivative is R(−)-10-{β-D-glucopyranosyl-}, 11-hydroxyaporphine.
